(19) United States
(12) Patent Application Publication
(10) Pub. No.: US 2017/0231840 A1
(43) Pub. Date: Aug. 17, 2017

(54) Adhesive Nursing Pad

(71) Applicant: Zaleatra Taylor, Orlando, FL (US)

(72) Inventor: Zaleatra Taylor, Orlando, FL (US)

(21) Appl. No.: 15/046,274

(22) Filed: Feb. 17, 2016

(51) Int. Cl. A61F 13/58; A61L 15/26; A61L 15/58; A41C 3/04; A41C 3/14

(52) U.S. Cl. CPC A61F 13/58; A41C 3/04; A41C 3/144; A61L 15/58; A61L 15/26; A61F 2013/15016

(57) ABSTRACT

This invention concerns an adhesive nursing pad particularly useful for absorbing body fluids. The adhesive nursing pad comprises of an inner absorbing layer with a silicone adhesive circumference that attaches to the woman's breast for securely maintaining the nursing pad's position without slippage upon leakage of fluids from the breast of nursing or pregnant women during non-nursing times and a moisture impermeable outer.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

Everyday women around the world become pregnant and give birth. Upon entering the final weeks or the first weeks of breast feeding, a woman's breast become tender, swollen, and in some cases full with milk. Once a baby is born, breast feeding takes place. The breasts continue to produce daily to supply the frequent feeding habits of the newborn, leaving the breast sore, and leaking milk. Such occurrences may result in leaving stains on the bed linen from the breast leaking overnight and at the very least will increase in required cleaning frequency of the woman's clothing.

It is of course, highly desirable to maintain the nursing pad securely in a fixed position over the woman's nipple. Adhesive materials have been applied to the moisture impermeable outer layer of the nursing pad to engage and adhere to an inner surface of the woman's garment, such as a regular or nursing brassiere. It is particularly difficult to maintain the nursing pad in proper position at night during sleep when leakage from the woman is likely to stain the woman's bed clothing and bedding materials such as sheets and covers. Some nursing pads currently available make use of an adhesive positioned on an outer surface, but are of limited use because of the location and area of application of the adhesive material on the pad. Current nursing pads are easily detached from a woman's garment resulting in movement of the pad from the woman's nipple and/or folding over of the pad on itself so that it no longer engages the nipple resulting in failure of the pad to perform its intended function. Furthermore, wearing a brassiere while pregnant is considered uncomfortable by many women and can often become a nuisance.

### BRIEF SUMMARY OF THE INVENTION

The present invention generally relates to an adhesive nursing pad for wear by nursing and pregnant women, which adheres to the breast of pregnant and nursing women. The invention specifically relates to an adhesive nursing pad with an inner adhesive circumference for maintaining the pad in a fixed position on the woman's nipple upon leakage of breast milk from the breast of nursing and pregnant women during non-nursing times.

### BRIEF DESCRIPTION OF THE DRAWINGS

FIG. 1 is a perspective view of the front of the adhesive nursing pad, an inner absorption layer with an adhesive circumference, and a moisture impermeable outer layer.

FIG. 2 is a perspective view of the side of the adhesive nursing pad, an inner absorption layer with an adhesive circumference, and a moisture impermeable outer layer.

### DETAILED DESCRIPTION OF THE INVENTION

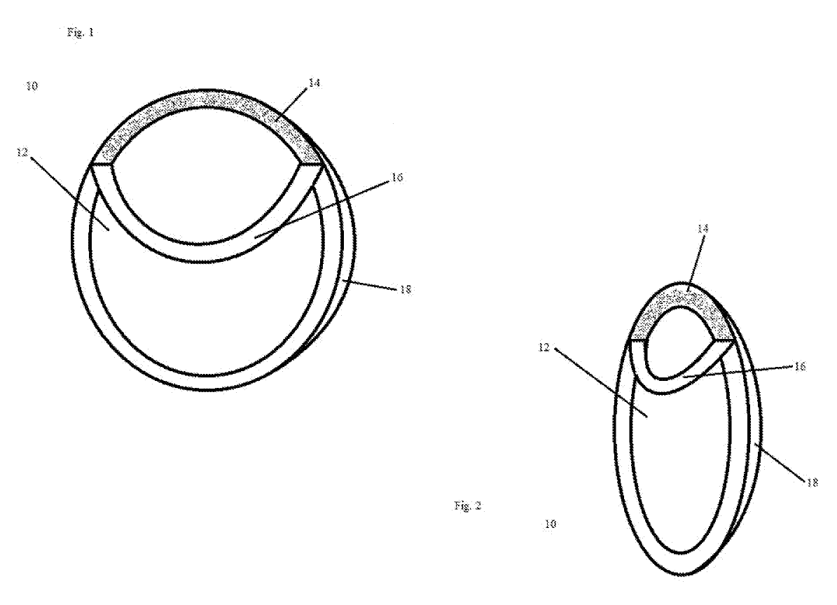

Referring now to the invention in more detail, in FIG. 1 and FIG. 2 there is shown a perspective view of an adhesive nursing pad 10, with a high absorption inner layer 12, which is placed in contact with a woman's lactating breast for absorbing leaked milk, and a moisture impermeable outer layer 18, usually positioned against the woman's clothing such as a regular or nursing brassiere.

In more detail the adhesive nursing pad 10 is generally circular in shape and is contoured so as to receive and engage the breast of a woman and cover her nipple. Thus, the inner surface of the adhesive nursing pad 10 as shown in FIG. 1 is generally concave, while its outer surface as shown in FIG. 2 is convex. The adhesive nursing pad 10 has a multi-layer structure and includes an inner absorbent layer 12, in contact with the woman's breast, and a moisture impermeable outer layer 18. The inner absorbent layer 12 and the moisture impermeable outer layer 18 may be bonded together by conventional means such as thermal bonding The inner absorbent layer 12 is preferably comprised of a non-woven fabric material which has a high coefficient of fluid absorbance. One well known fiber material commonly used in these types of applications is comprised of copolymer micro-fibers. The inner absorbent layer 12 may itself include an inner layer in contact with the woman's breast which allows the woman's milk to pass into and be absorbed by the adhesive nursing pad's inner absorbent layer 12. The moisture impermeable outer layer 18 is preferably comprised of a moisture impermeable material and is positioned against the woman's clothing such as a regular or nursing brassiere (not shown for simplicity). The moisture impermeable outer layer 18 may be conventional in composition and structure and preferably is in the form of a web of non-woven fibers preferably treated with a laminating material for rendering the moisture impermeable outer layer 18 moisture impermeable so as to prevent the woman's milk from passing through the adhesive nursing breast pad 10 and staining the woman's clothes and bedding. An example of a laminating material which could be used for the moisture impermeable outer layer 18, of the adhesive nursing breast pad 10, is a thin material treated with a light plastic or rubber latex to render the thin layer moisture impermeable. The structure and configuration of the adhesive nursing pad 10 described thus far is conventional and well-known to those familiar with disposable breast pads.

In further detail with several aspects of the present invention of the adhesive nursing pad 10, positioned on the inner absorbent layer's 12 circumference, is an approved medical grade silicone adhesive 14. The silicone adhesive 14 is positioned on the circumference of the inner absorbent layer 12 to be attached to the woman's breast. The purpose of the inner absorbent layer 12 is to absorb discharged milk and carry it away from the woman's breast. The purpose of the silicone adhesive 14 positioned on the inner absorbent layer's 12 circumference, is to attach to the woman's breast securely maintaining the adhesive nursing pad's 10 position on the breast of nursing or pregnant women, upon leakage of fluids during non-nursing times. The purpose of the moisture impermeable outer layer 18 is the prevent staining of the woman's brassier or clothing.

Referring to FIG. 1 and FIG. 2, there is shown a perspective view of the adhesive nursing pad 10 in accordance with the present invention. As in the previously described embodiment, the adhesive nursing pad 10 includes an inner absorbent layer 12, with silicone adhesive 14 positioned on the inner absorbent's layer 12 circumference, a moisture impermeable outer layer 18. Positioned over the silicone adhesive 14 is a covering layer 16 for protecting the silicone adhesive 14 prior to use. As shown in FIG. 1 and FIG. 2, a covering layer 16 is shown, slightly bent backwards. It being understood to be positioned over the silicone adhesive 14 prior to use. The covering layer 16 protects the silicone adhesive 14 prior to use and maintains it in a tacky condition. The covering layer 16 is adapted for manual grasping and pulling off of the silicone adhesive 14 and is well known in these types of adhesive items.

The advantages of the present invention include, without limitation, that it will prevent constant breast milk leakage through the brassiere and clothing during the night when the breast tends to shift around the brassiere. Further, a pregnant or nursing mom not caring to wear a brassier because of tender and sore nipples can remove her brassier leaving the adhesive nursing pad, being that it will be attached to the breast and not the brassiere.

In broad embodiment, the present invention is a nursing pad that adheres directly to the mother's breast.

While the foregoing written description of the invention enable one of ordinary skill to make and use what is considered presently to be the best mode thereof, those of ordinary skill will understand and appreciate the existence of variations, combinations, and equivalents of the specific, embodiment, method, and examples herein. The invention should therefore not be limited by the above described embodiment, method, and examples, but by all embodiments and methods within the scope and spirit of the invention as claimed.

1. An adhesive nursing pad, comprising of:
a generally circular member having a contoured shape for engaging a woman's breast, said member including a moisture absorbing inner layer and a moisture impermeable moisture impermeable outer layer;
a silicone adhesive positioned along said member's moisture absorbing inner layer's circumference, said silicone adhesive being configured to attach to the woman's breast securely maintaining said member's position on the woman's breast during non-nursing times;

2. The nursing pad of claim 1, further comprising; a removable covering that is positioned across an entirety of the said silicone adhesive.

3. A adhesive nursing pad, comprising;
a generally circular member having a contoured shape for engaging a woman's breast, said member including a moisture absorbing inner layer and a moisture impermeable moisture impermeable outer layer;
a silicone adhesive positioned along said member's moisture absorbing inner layer's circumference, said silicone adhesive being configured to attach to the woman's breast securely maintaining said member's position on the woman's breast during non-nursing times.
